(19) United States
(12) Patent Application Publication
(10) Pub. No.: US 2026/0202335 A1
(43) Pub. Date: Jul. 16, 2026

(54) Test Device and Method for Rapidly Determining Factors Affecting Water Transparency

(71) Applicant: Shanghai Investigation, Design & Research Institute Co., Ltd., Shanghai (CN)

(72) Inventors: Wei HU, Shanghai (CN); Kuo GAO, Shanghai (CN); Yali GUO, Shanghai (CN); Ning FANG, Shanghai (CN)

(73) Assignee: Shanghai Investigation, Design & Research Institute Co., Ltd., Shanghai (CN)

(21) Appl. No.: 18/702,795

(22) PCT Filed: Mar. 28, 2024
(86) PCT No.: PCT/CN2024/084259

§ 371 Date: Mar. 30, 2026

(30) Foreign Application Priority Data
Jun. 1, 2023 (CN) 202310647527.2

(51) Int. Cl. G01N 21/59; G01N 1/34; G01N 1/38; G01N 33/18

(52) U.S. Cl. CPC G01N 21/59; G01N 1/34; G01N 1/38; G01N 33/1893; G01N 2201/061

(57) ABSTRACT

A test device and a method for rapidly determining factors affecting water transparency are provided. The test device comprises a testing pool, a light source, an illumination sensor, an intermediate pool, a water component separating assembly, a stirring assembly and a control system assembly. The light source is above the testing pool. The illumination sensor is at a lower center of the testing pool. The testing pool is communicated with the intermediate pool through pipelines. The illumination sensor measures the transparency of water samples containing different components to determine the influence of each on water transparency. This approach addresses the challenges in quickly diagnosing the causes of low water transparency and analyzing the impact of sudden pollution events with existing technology. Consequently, this test device helps in swiftly identifying the main pollution factors in low-transparency water bodies and provides crucial support for the precise management of such water bodies.

### FIELD OF THE INVENTION

The present disclosure relates to the technical field of water environment remediation equipment and processes, in particular to a test device and a method for rapidly determining factors affecting water transparency.

### BACKGROUND OF THE INVENTION

Current water environment management is transitioning from focusing solely on basic water quality improvement to exploring new goals, such as aquatic environment restoration, harmony between humans and water, and achieving clear water with green shores. However, numerous waterways still suffer from issues like turbidity, low transparency, and poor visual quality due to suspended solids, organic debris, and algae accumulation, resulting in a significant gap between the current state and the high-quality water environment and healthy river-lake systems we envision. Enhancing water transparency is becoming a crucial focus in the next phase of water management, following the initial improvements in water quality. Research on the factors influencing water transparency is essential for transparency enhancement and holds significant importance.

Water transparency is primarily influenced by particles and dissolved colored substances in the water. “Particles” generally refer to entities with a diameter greater than 0.45 micrometers, including large suspended particles (such as silt and mineral particles), small inorganic suspended particles (such as clay particles and some metal salts), and algae. Large suspended particles (with a diameter greater than 70 micrometers) can naturally settle in a short time and their states are mainly influenced by the water body's dynamics. Human activity or wind and waves can disturb these particles, affecting water transparency. Small inorganic suspended particles (with a diameter less than 70 micrometers) primarily include metal hydroxide colloids and clay particles, which are difficult to settle quickly and are affected by incoming water quality and the surrounding environment. Additionally, common algae in surface water bodies range from a few micrometers to several tens of micrometers in size, are primarily influenced by water nutrients, and exhibit seasonal variations. Dissolved colored substances (with a diameter less than 0.45 micrometers mainly include colored dissolved organic matter and metal salts. These substances are related to incoming water quality and the surrounding environment.

Currently, the detection of water transparency mainly relies on two methods: Secchi disks and transparency meters. These methods involve visual observation of specific underwater markers by testing personnel, leading to significant subjective errors and an inability to reflect subtle changes in transparency. Moreover, diagnosing the causes of low water transparency currently relies on long-term monitoring data accumulation and statistical analysis to determine the correlation between different water quality indicators and water transparency, requiring significant manpower and resources for on-site detection. Consequently, it is challenging to quickly identify factors affecting water transparency and to analyze the impact of sudden pollution events. Given the above, it is urgent to develop a test device and a method for rapidly determining the factors affecting water transparency.

### SUMMARY OF THE INVENTION

In view of the above-mentioned shortcomings, the present disclosure provides a test device and a method for rapidly determining factors affecting water transparency, which address the issue where existing methods require significant manpower and resources for on-site detection, making it difficult to swiftly identify the factors affecting water transparency.

A first embodiment of the present disclosure provides a test device for rapidly determining factors affecting water transparency, comprising a testing pool, a light source, an illumination sensor, an intermediate pool, a water component separating assembly, a stirring assembly and a control system assembly. The light source is disposed above the testing pool, and the illumination sensor is disposed at a lower center of the testing pool. The testing pool is communicated with the intermediate pool through a first pipeline and a second pipeline, a first end of the first pipeline is connected with an upper end of the testing pool through a first communication valve, and a second end of the first pipeline is connected with a lower end of the intermediate pool through a second communication valve. A first end of the second pipeline is connected with a lower end of the testing pool through a third communication valve, and a second end of the second pipeline is connected with an upper end of the intermediate pool through a fourth communication valve. The water component separating assembly is disposed at the second pipeline and is configured to separate components in water samples. Each of the first pipeline and the second pipeline is provided with a driving pump. The stirring assembly is disposed within the intermediate pool and is configured to evenly mix water samples in the intermediate pool. The illumination sensor is in communication with the control system assembly.

Preferably, the lower end of the testing pool is further provided with a first emptying valve for draining water samples in the testing pool. The lower end of the intermediate pool is provided with a second emptying valve for draining the water samples in the intermediate pool, the upper end of the intermediate pool is provided with a normally-open communication valve for communicating with atmospheric environment, and oxidant is added into the intermediate pool through the normally-open communication valve.

Preferably, the testing pool is cylindrical, a ratio of an inner diameter of the testing pool to its depth is within a range between ⅛ and 1/15, and a depth of the water samples in the testing pool is at least two-thirds of the depth of the testing pool. The testing pool is made of opaque materials.

Preferably, the light source is a xenon lamp, whose power is within a range between 35 W and 45 W.

Preferably, a scale of the illumination sensor is from 0 lux to 60000 lux.

Preferably, the water component separating assembly comprises an upper shell, a lower shell, and a filter. The upper shell and the lower shell are connected through threads, a filter supporting layer is disposed within the lower shell, through holes having a fixed aperture are formed in the filter supporting layer, and the filter is disposed on the filter supporting layer. The upper shell is provided with a sealing ring, and the sealing ring is configured to press the filter against the filter supporting layer.

Preferably, the stirring assembly comprises a motor and a stirring paddle. The motor is disposed at a center of a top end face of the intermediate pool, the stirring paddle is fixed to a shaft of the motor, and the motor drives the stirring paddle to evenly mix the water samples in the intermediate pool.

Preferably, the motor is a brushless motor, whose speed is in a range between 10 r/min and 800 r/min.

Preferably, the driving pump is a peristaltic pump, whose pressure is in a range between 0.1 MPa and 0.2 Mpa.

A second embodiment of the present disclosure provides a method for using a test device for rapidly determining factors affecting water transparency as described in any one of the examples provided in the first embodiment of the present disclosure. The lower end of the testing pool is further provided with a first emptying valve for draining water samples in the testing pool. The upper end of the intermediate pool is provided with a normally-open communication valve for communicating with atmospheric environment, and oxidant is added into the intermediate pool through the normally-open communication valve. A filter is provided within the water component separating assembly. The present disclosed method comprises the following steps.

Step S1 comprises: testing a water transparency of original water samples, and adding the original water samples into the testing pool; turning on the light source, measuring an illuminance (Ix1) by the illumination sensor, and feeding back data of the illuminance (Ix1) to the control system assembly.

Step S2 comprises: testing a water transparency of water samples after filtering out large suspended particles.

Step S2.1 comprises: disposing a filter with an aperture of 75 μm within the water component separating assembly; turning on the third communication valve, the fourth communication valve, and the driving pump on the second pipeline, at which time the water samples in the testing pool pass through the filter and enter the intermediate pool.

Step S2.2 comprises: turning on the first communication valve, the second communication valve, and the driving pump on the first pipeline, at which time the water samples in the intermediate pool enter the testing pool; measuring an illuminance (Ix2) by the illumination sensor, and feeding back data of the illuminance (Ix2) to the control system assembly.

Step S3 comprises: testing a water transparency of water samples after filtering out small inorganic suspended particles.

Step S3.1 comprises: replacing the filter with the aperture of 75 μm within the water component separating assembly with a filter with an aperture of 0.45 μm; turning on the third communication valve, the fourth communication valve, and the driving pump on the second pipeline, at which time the water samples in the testing pool pass through the filter with an aperture of 0.45 μm and enter the intermediate pool.

Step S3.2 comprises: turning on the first communication valve, the second communication valve, and the driving pump on the first pipeline, at which time the water samples in the intermediate pool enter the testing pool; measuring an illuminance (Ix3) by the illumination sensor; and feeding back data of the illuminance (Ix3) to the control system assembly.

Step S4 comprises: filtering out algae; and testing a water transparency of water samples containing only the small inorganic suspended particles.

Step S4.1 comprises: turning on the first emptying valve to drain the water samples in the testing pool; adding oxidant having the same volume as the drained water samples into the testing pool; measuring an illuminance (Ix0) by the illumination sensor; and feeding back data of the illuminance (Ix0) to the control system assembly.

Step S4.2 comprises: inverting the filter with an aperture of 0.45 μm within the water component separating assembly; turning on the third communication valve, the fourth communication valve, and the driving pump on the second pipeline, at which time the oxidant in the testing pool passes through the inverted filter and enter the intermediate pool, such that filtered-out substances on the inverted filter are flushed into the intermediate pool.

Step S4.3 comprises: evenly mixing the oxidant in the intermediate pool by the stirring assembly such that filtered-out algae fully react with the oxidant; turning on the first communication valve, the second communication valve, and the driving pump on the first pipeline; configuring the oxidant in the intermediate pool to enter the testing pool; measuring an illuminance (Ix4) by the illumination sensor; and feeding back data of the illuminance (Ix4) to the control system assembly.

Step S5 comprises: calculating, by the control system assembly, an impact of each of the components in the water samples on water transparency based on data obtained from S1-S4, where a percentage of water transparency degradation that the large suspended particles account for is given by:

$$\frac{lx 2 - lx 1}{lx 0 - lx 1} ;$$

a percentage of water transparency degradation that the small inorganic suspended particles account for is given by:

$$\frac{lx 0 - lx 4}{lx 0 - lx 1} ;$$

a percentage of water transparency degradation that the dissolved colored substances account for is given by:

$$\frac{lx 0 - lx 3}{lx 0 - lx 1} ;$$

and a percentage of water transparency degradation that the algae account for is given by:

$$\frac{( lx 3 - lx 2 ) - ( lx 0 - lx 4 )}{lx 0 - lx 1} .$$

As described above, the present disclosed test device and method have the following advantages:

By incorporating the water component separating assembly, utilizing the filter within the water component separating assembly, and adding the oxidant into the water samples, the present disclosed test device and method sequentially isolate the components that affect water transparency, such as large suspended particles, small inorganic suspended particles, algae, and dissolved colored substances. The illumination sensor then measures the transparency of water samples containing these different components, determining the influence of each on water transparency. This approach addresses the challenges in quickly diagnosing the causes of low water transparency and analyzing the impact of sudden pollution events. Consequently, the presently disclosed test device helps in swiftly identifying the main pollution factors in low-transparency water bodies and provides crucial support for the precise management of such water bodies.

### BRIEF DESCRIPTION OF DRAWINGS

FIG. 1 is a schematic structural diagram of a test device for rapidly determining factors affecting water transparency according to the present disclosure.

### REFERENCE NUMERALS

1. Testing pool; 2. Water component separating assembly; 3. Intermediate pool; 4. Light source; 5. Illumination sensor; 6. Stirring assembly; 7. Control system assembly; 8. First communication valve; 9. Second communication valve; 10. Third communication valve; 11. Fourth communication valve; 12. First emptying valve; 13. Second emptying valve; 14. Normally-open communication valve; 15. Driving pump.

### DETAILED DESCRIPTION OF THE INVENTION

The embodiments of the present disclosure will be described below. Those skilled can easily understand disclosure advantages and effects of the present disclosure according to contents disclosed by the specification.

It should be understood that the structures, proportions, sizes, and the like, which are illustrated in the drawings of the present specification, are only used to clarify the contents disclosed in the specification for understanding and reading by those skilled, and are not intended to limit the implementation of the present disclosure, thus are not technically meaningful. Any modification of the structure, change of the scale, or adjustment of the size should still fall within the scope of the technical contents disclosed by the present disclosure without affecting the effects and achievable objectives of the present disclosure. Terms such as “upper”, “lower”, “left”, “right”, “middle”, and “a” used in this specification are only for ease of description, and they are not intended to restrict the scope of implementation of the present invention. Any change or adjustment of corresponding relative relationships without any substantial technical change should be regarded as within the scope of the implementation of the present disclosure.

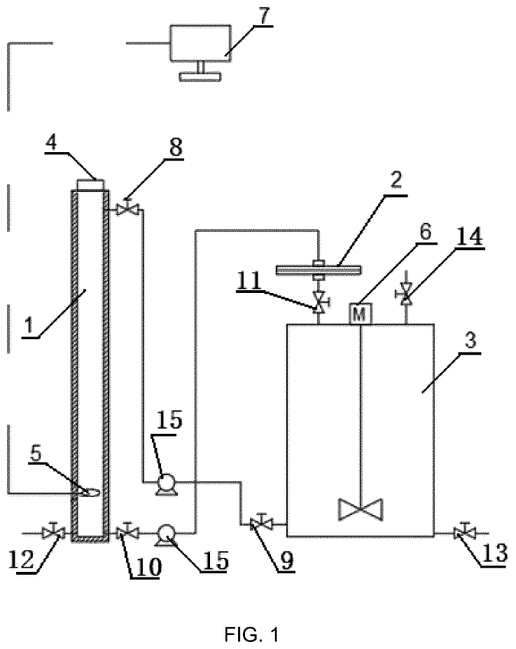

As shown in FIG. 1, the present disclosure provides a test device for rapidly determining factors affecting water transparency. The test device comprises a testing pool 1, a light source 4, an illumination sensor 5, an intermediate pool 3, a water component separating assembly 2, a stirring assembly 6 and a control system assembly 7. The light source 4 is disposed above the testing pool 1, and the illumination sensor 5 is disposed at a lower center of the testing pool 1. The testing pool 1 is communicated with the intermediate pool 3 through a first pipeline and a second pipeline. A left end (or, first end) of the first pipeline is connected with an upper end of the testing pool 1 through a first communication valve 8, and a right end (or, second end) of the first pipeline is connected with a lower end of the intermediate pool 3 through a second communication valve 9. A left end (or, first end) of the second pipeline is connected with a lower end of the testing pool 1 through a third communication valve 10, and a right end (or, second end) of the second pipeline is connected with an upper end of the intermediate pool 3 through a fourth communication valve 11. The water component separating assembly 2 is disposed at the second pipeline and is configured to separate components in water samples. Each of the first pipeline and the second pipeline is provided with a driving pump 15. The stirring assembly 6 is disposed within the intermediate pool 3 and is configured to evenly mix water samples in the intermediate pool 3. The illumination sensor 5 is in communication with the control system assembly 7.

By incorporating the testing pool 1, the intermediate pool 3, and the water component separating assembly 2, and connecting the testing pool 1 with the intermediate pool 3 through the first pipeline and the second pipeline, the presently disclosed test device controls the on-off state of these pipelines, the filtration effiency of the filter within the water component separating assembly, and the bleaching method using oxidants, to sequentially isolate components that affect water transparency, such as large suspended particles, small inorganic suspended particles, algae, and dissolved colored substances. Subsequently, the illumination sensor 5 measures the transparency of water samples containing these different components, determining the influence of each on water transparency, thereby simplifying the method while ensuring precise measurement results.

Preferably, as shown in FIG. 1, the lower end of the testing pool 1 is further provided with a first emptying valve 12 for draining water samples in the testing pool 1. The lower end (or, bottom end) of the intermediate pool 3 is provided with a second emptying valve 13 for draining water samples in the intermediate pool 3, the upper end (or, top end) of the intermediate pool 3 is provided with a normally-open communication valve 14 for communicating with atmospheric environment, and oxidant is added into the intermediate pool 3 through the normally-open communication valve 14. In one embodiment, the volume of the intermediate pool 3 is determined based on the maximum volume of the testing pool 1: When the entire water samples from the testing pool 1 are transferred to the intermediate pool 3, the depth of the water samples in the intermediate pool 3 is not less than one-third of the total depth of the intermediate pool 3.

Additionally, the present disclosed test device can utilize either sodium hypochlorite or hydrogen peroxide as the oxidant. The dosage (or concentration) of these oxidants and the reaction time with algae are detailed in Table 1 below:

TABLE 1
Concentration and Reaction Time of Oxidants

| | | Reaction |
|---|---|---|
| Types of Oxidants | Dosage | Time |
| Sodium Hypochlorite | 10~30 mg/L (effective chlorine content) | 5~10 min |
| Hydrogen Peroxide | 20%~35% (volume fraction) | 5~10 min |

Preferably, as shown in FIG. 1, the testing pool 1 is cylindrical, a ratio of an inner diameter of the testing pool 1 to its depth is within a range between ⅛ and 1/15, and a depth of the water samples in the testing pool 1 is at least two-thirds of the depth of the testing pool 1. The testing pool 1 is made of opaque materials. In one embodiment, the testing pool 1 can be made of stainless steel or gray Unplasticized Polyvinyl Chloride (UPVC) pipes to prevent interference from external light. The light source 4 is a xenon lamp, whose power is within a range between 35 W and 45 W. The illumination sensor 5 has a detection scale of 0 to 60000 lux, and communicates the measurement data back to the control system assembly 7 through an RS485 serial port.

Preferably, as shown in FIG. 1, the water component separating assembly 2 comprises an upper shell, a lower shell, and a filter. The upper shell and the lower shell are connected through threads, a filter supporting layer is disposed within the lower shell, through holes having a fixed aperture are formed in the filter supporting layer, and the filter is disposed on the filter supporting layer. The upper shell is provided with a sealing ring, and the sealing ring is configured to press the filter against the filter supporting layer. Furthermore, the aperture range of the through holes on the filter supporting layer is between 70 μm and 1 mm, and the filter supporting layer is made of stainless steel. The sealing ring uses an O-ring. Additionally, in one embodiment, considering the chemical resistance and cost-effectiveness of the filter, the filter can be a nylon filter screen with an aperture of 75 μm (200 mesh) or a polyvinylidene fluoride (PVDF) separation membrane with an aperture of 0.45 μm. The 75-μm nylon filter screen is primarily used to filter large suspended particles, while the 0.45-μm PVDF separation membrane is mainly used to filter algae and small inorganic suspended particles.

Preferably, as shown in FIG. 1, the stirring assembly 6 comprises a motor and a stirring paddle. The motor is disposed at a center of a top end face of the intermediate pool 3, the stirring paddle is fixed to a shaft of the motor, and the motor drives the stirring paddle to evenly mix the water samples in the intermediate pool 3. In one embodiment, the motor is a brushless motor, whose speed is in a range between 10 r/min and 800 r/min. The driving pump 15 is a peristaltic pump, whose pressure is in range between 0.1 MPa and 0.2 Mpa. The operating pressure of the water component separating assembly 2 is provided by the driving pump 15.

Preferably, in one embodiment, the control system assembly 7 uses a computer.

The present disclosure further provides a method for using a test device for rapidly determining factors affecting water transparency as described in any one of the above embodiments of the present disclosure. The lower end of the testing pool 1 is further provided with a first emptying valve 12 for draining water samples in the testing pool 1. The upper end of the intermediate pool 3 is provided with a normally-open communication valve 14 for communicating with atmospheric environment, and oxidant is added into the intermediate pool 3 through the normally-open communication valve 14. A filter is provided within the water component separating assembly 2. The present disclosed method comprises the following steps.

Step A1 comprises: testing a water transparency of original water samples.

First, a specific volume of the original water samples is added into the testing pool 1, the light source 4 is then turned on, the illumination sensor 5 measures an illuminance (Ix1), and data of the illuminance (Ix1) is fed back to the control system assembly 7. The data of the illuminance (Ix1) represents the combined effect of large suspended particles, small inorganic suspended particles, algae, and dissolved colored substances on the water transparency.

Step A2 comprises: testing a water transparency of water samples after filtering out large suspended particles.

Step A2.1 comprises: disposing a filter with an aperture of 75 μm within the water component separating assembly 2; turning on the third communication valve 10, the fourth communication valve 11, and the driving pump 15 on the second pipeline, at which time the water samples in the testing pool 1 in step A1 pass through the filter and enter the intermediate pool 3. The filter filters out the large suspended particles, leaving the water samples in the intermediate pool 3 containing small inorganic suspended particles, algae, and dissolved colored substances.

Step A2.2 comprises: turning on the first communication valve 8, the second communication valve 9, and the driving pump 15 on the first pipeline, at which time the water samples in the intermediate pool 3 enter the testing pool 1; measuring an illuminance (Ix2) by the illumination sensor 5; and feeding back data of the illuminance (Ix2) to the control system assembly 7. The data of the illuminance (Ix2) represents the combined effect of small inorganic suspended particles, algae, and dissolved colored substances on the water transparency.

Step A3 comprises: testing a water transparency of water samples after filtering out small inorganic suspended particles.

Step c comprises: removing the filter with the aperture of 75 μm used in step A2.1 from the water component separating assembly 2, and rinsing the water component separating assembly 2 with deionized water; after rinsing, placing a filter with an aperture of 0.45 μm into the water component separating assembly 2, turning on the third communication valve 10, the fourth communication valve 11, and the driving pump 15 on the second pipeline, at which time the water samples in the testing pool 1 in step A2.2 pass through the filter with an aperture of 0.45 μm and enter the intermediate pool 3. The filter with an aperture of 0.45 μm filters out small inorganic suspended particles and algae, leaving the water samples in the intermediate pool 3 containing only dissolved colored substances.

Step A3.2 comprises: turning on the first communication valve 8, the second communication valve 9, and the driving pump 15 on the first pipeline, at which time the water samples in the intermediate pool 3 enter the testing pool 1; measuring an illuminance (Ix3) by the illumination sensor 5; and feeding back data of the illuminance (Ix3) to the control system assembly 7. The data of the illuminance (Ix3) represents the effect of dissolved colored substances on water transparency.

Step A4 comprises: filtering out algae, and testing a water transparency of water samples containing only the small inorganic suspended particles.

Step A4.1 comprises: turning on the first emptying valve 12 to drain the water samples in the testing pool 1 in step A3.2; adding oxidant having the same volume as the water samples entering the testing pool 1 in step A3.2 into the testing pool 1; measuring an illuminance (Ix0) by the illumination sensor 5; and feeding back data of the illuminance (Ix0) to the control system assembly 7. The data of the illuminance (Ix0) represents the transparency of the oxidant, which is generally a constant value.

Step A4.2 comprises: inverting the filter with an aperture of 0.45 μm used in step A3.1 and placing it back into the water component separating assembly 2; turning on the third communication valve 10, the fourth communication valve 11, and the driving pump 15 on the second pipeline, at which time the oxidant in the testing pool 1 pass through the inverted filter and enter the intermediate pool 3, such that filtered-out substances on the inverted filter are flushed into the intermediate pool 3.

Step A4.3 comprises: activating the stirring assembly 6; configuring the motor to drive the stirring paddle to evenly mix the oxidant, algae, and small inorganic suspended particles in the intermediate pool 3, such that the algae reacts fully with the oxidant (reaction times for different oxidants vary, see Table 1 for specifics); turning on the first communication valve 8, the second communication valve 9, and the driving pump 15 on the first pipeline; configuring the oxidant in the intermediate pool 3 to enter the testing pool 1; measuring an illuminance (Ix4) by the illumination sensor 5; and feeding back data of the illuminance (Ix4) to the control system assembly 7. The data of the illuminance (Ix4) represents the effect of small inorganic suspended particles on water transparency.

Step A5 comprises: calculating, by the control system assembly 7, an impact of each of the components in the water samples on water transparency based on data obtained from A1-A4, where a percentage of water transparency degradation that the large suspended particles account for is given by:

$$\frac{lx 2 - lx 1}{lx 0 - lx 1} ;$$

a percentage of water transparency degradation that the small inorganic suspended particles account for is given by:

$$\frac{lx 0 - lx 4}{lx 0 - lx 1} ;$$

a percentage of water transparency degradation that the dissolved colored substances account for is given by:

$$\frac{lx 0 - lx 3}{lx 0 - lx 1} ;$$

and a percentage of water transparency degradation that the algae account for is given by:

$$\frac{( lx 3 - lx 2 ) - ( lx 0 - lx 4 )}{lx 0 - lx 1} .$$

Furthermore, in steps A1 to A4, after each instance of guiding the water samples from the testing pool 1 to the intermediate pool 3 or from the intermediate pool 3 to the testing pool 1 using the first communication valve 8, the second communication valve 9, the third communication valve 10, the fourth communication valve 11, the driving pump 15, and/or the first emptying valve 12, the related valves and the pump should be turned off. The first communication valve 8 and the second communication valve 9 are located on the first pipeline, while the third communication valve 10 and the fourth communication valve 11 are located on the second pipeline.

Based on the analysis of two sets of data, the factors affecting water transparency are examined as follows. The light source 4 is a 45 W xenon lamp. The testing pool 1 has a diameter of 40 mm and a height of 600 mm. The oxidant used is a 15 mg/L sodium hypochlorite solution. The filter comprises the 0.45-μm PVDF separation membrane and the 75-μm nylon filter screen. The volume of the original water samples is 500 mL. The test data is shown in Table 2 below.

TABLE 2
Test Results

| | Test 1 | Test 2 |
|---|---|---|
| Illuminance | Samples From Inland | Samples From Enclosed |
| (lx) | Waterway | Waters in a Polder Area |
| lx0 | 5.0 | 5.0 |
| lx1 | 1.5 | 1.3 |
| lx2 | 4.6 | 3.5 |
| lx3 | 4.9 | 4.8 |
| lx4 | 4.8 | 4.7 |

Based on the above data and formulas, the impact of different components in the water samples on water transparency can be calculated as follows.

For the samples from the inland waterway: large suspended particles account for 88.57%, small inorganic suspended particles for 5.71%, dissolved colored substances for 2.86%, and algae for 2.86%.

For the samples from the enclosed waters in the polder area: large suspended particles account for 59.46%, small inorganic suspended particles for 8.11%, dissolved colored substances for 5.41%, and algae for 27.03%.

By incorporating the testing pool 1 and the intermediate pool 3, connecting the testing pool 1 with the intermediate pool 3 through the pipelines, arranging the water component separating assembly 2 on the pipelines, and disposing the illumination sensor 5 within the testing pool 1, the present disclosed test device sequentially isolates components that affect water transparency, such as large suspended particles, small inorganic suspended particles, algae, and dissolved colored substances, through the filter and the oxidants. Subsequently, the illumination sensor 5 measures the transparency of water samples containing these different components, determining the influence of each on water transparency. Using an illumination sensor 5 for measuring water transparency provides high accuracy and reproducibility, effectively solving the challenges of the current methods that require significant manpower and resources for on-site detection with Secchi disks and transparency meters, which cannot quickly determine the factors affecting water transparency.

Therefore, the present disclosure effectively overcomes various shortcomings in the existing technology and has high industrial utilization value.

The above-mentioned embodiments are for exemplarily describing the principle and effects of the present disclosure instead of limiting the present disclosure. Those skilled in the art can make modifications or changes to the above-mentioned embodiments without going against the spirit and the range of the present disclosure. Therefore, all equivalent modifications or changes made by those who have common knowledge in the art without departing from the spirit and technical concept disclosed by the present disclosure shall be still covered by the scope of the present disclosure.

1. A test device for rapidly determining factors affecting water transparency, comprising a testing pool (1), a light source (4), an illumination sensor (5), an intermediate pool (3), a water component separating assembly (2), a stirring assembly (6) and a control system assembly (7), wherein the light source (4) is disposed above the testing pool (1), and the illumination sensor (5) is disposed at a lower center of the testing pool (1); wherein the testing pool (1) is communicated with the intermediate pool (3) through a first pipeline and a second pipeline, a first end of the first pipeline is connected with an upper end of the testing pool (1) through a first communication valve (8), and a second end of the first pipeline is connected with a lower end of the intermediate pool (3) through a second communication valve (9); wherein a first end of the second pipeline is connected with a lower end of the testing pool (1) through a third communication valve (10), and a second end of the second pipeline is connected with an upper end of the intermediate pool (3) through a fourth communication valve (11); wherein the water component separating assembly (2) is disposed at the second pipeline and is configured to separate components in water samples; wherein the water component separating assembly (2) comprises an upper shell, a lower shell, and a filter, wherein the upper shell and the lower shell are connected through threads, a filter supporting layer is disposed within the lower shell, through holes having a fixed aperture are formed in the filter supporting layer, and the filter is disposed on the filter supporting layer; wherein the upper shell is provided with a sealing ring, and the sealing ring is configured to press the filter against the filter supporting layer; wherein each of the first pipeline and the second pipeline is provided with a driving pump (15); wherein the stirring assembly (6) is disposed within the intermediate pool (3) and is configured to evenly mix water samples in the intermediate pool (3); wherein the illumination sensor (5) is in communication with the control system assembly (7).

2. The test device according to claim 1, wherein the lower end of the testing pool (1) is further provided with a first emptying valve (12) for draining water samples in the testing pool (1); wherein the lower end of the intermediate pool (3) is provided with a second emptying valve (13) for draining the water samples in the intermediate pool (3), the upper end of the intermediate pool (3) is provided with a normally-open communication valve (14) for communicating with atmospheric environment, and oxidant is added into the intermediate pool (3) through the normally-open communication valve (14).

3. The test device according to claim 1, wherein the testing pool (1) is cylindrical, a ratio of an inner diameter of the testing pool (1) to its depth is within a range between ⅛ and 1/15, and a depth of the water samples in the testing pool (1) is at least two-thirds of the depth of the testing pool (1); wherein the testing pool (1) is made of opaque materials.

4. The test device according to claim 1, wherein the light source (4) is a xenon lamp, whose power is within a range between 35 W and 45 W.

5. The test device according to claim 1, wherein a scale of the illumination sensor (5) is from 0 lux to 60000 lux.

6. The test device according to claim 1, wherein the stirring assembly (6) comprises a motor and a stirring paddle, wherein the motor is disposed at a center of a top end face of the intermediate pool (3), the stirring paddle is fixed to a shaft of the motor, and the motor drives the stirring paddle to evenly mix the water samples in the intermediate pool (3).

7. The test device according to claim 6, wherein the motor is a brushless motor, whose speed is in a range between 10 r/min and 800 r/min.

8. The test device according to claim 1, wherein the driving pump (15) is a peristaltic pump, whose pressure is in a range between 0.1 MPa and 0.2 Mpa.

9. A method for using a test device for rapidly determining factors affecting water transparency according to claim 1, wherein the lower end of the testing pool (1) is further provided with a first emptying valve (12) for draining water samples in the testing pool (1), and a filter is provided within the water component separating assembly (2), wherein the method comprises:
S1: testing a water transparency of original water samples;
adding the original water samples into the testing pool (1), turning on the light source (4), measuring an illuminance (Ix1) by the illumination sensor (5), and feeding back data of the illuminance (Ix1) to the control system assembly (7);
S2: testing a water transparency of water samples after filtering out large suspended particles;
S2.1: disposing a filter with an aperture of 75 μm within the water component separating assembly (2), turning on the third communication valve (10), the fourth communication valve (11), and the driving pump (15) on the second pipeline, at which time the water samples in the testing pool (1) pass through the filter and enter the intermediate pool (3);
S2.2: turning on the first communication valve (8), the second communication valve (9), and the driving pump (15) on the first pipeline, at which time the water samples in the intermediate pool (3) enter the testing pool (1), measuring an illuminance (Ix2) by the illumination sensor (5), and feeding back data of the illuminance (Ix2) to the control system assembly (7);
S3: testing a water transparency of water samples after filtering out small inorganic suspended particles;
S3.1: replacing the filter with the aperture of 75 μm within the water component separating assembly (2) with a filter with an aperture of 0.45 μm, turning on the third communication valve (10), the fourth communication valve (11), and the driving pump (15) on the second pipeline, at which time the water samples in the testing pool (1) pass through the filter with an aperture of 0.45 μm and enter the intermediate pool (3);
S3.2: turning on the first communication valve (8), the second communication valve (9), and the driving pump (15) on the first pipeline, at which time the water samples in the intermediate pool (3) enter the testing pool (1), measuring an illuminance (Ix3) by the illumination sensor (5), and feeding back data of the illuminance (Ix3) to the control system assembly (7);
S4: filtering out algae, and testing a water transparency of water samples containing only the small inorganic suspended particles;
S4.1: turning on the first emptying valve (12) to drain the water samples in the testing pool (1), adding oxidant having the same volume as the drained water samples into the testing pool (1), measuring an illuminance (Ix0) by the illumination sensor (5), and feeding back data of the illuminance (Ix0) to the control system assembly (7);
S4.2: inverting the filter with an aperture of 0.45 μm within the water component separating assembly (2), turning on the third communication valve (10), the fourth communication valve (11), and the driving pump (15) on the second pipeline, at which time the oxidant in the testing pool (1) pass through the inverted filter and enter the intermediate pool (3), such that filtered-out substances on the inverted filter are flushed into the intermediate pool (3);
S4.3: evenly mixing the oxidant in the intermediate pool (3) by the stirring assembly (6) such that filtered-out algae fully react with the oxidant, turning on the first communication valve (8), the second communication valve (9), and the driving pump (15) on the first pipeline, configuring the oxidant in the intermediate pool (3) to enter the testing pool (1), measuring an illuminance (Ix4) by the illumination sensor (5), and feeding back data of the illuminance (Ix4) to the control system assembly (7);
S5: calculating, by the control system assembly (7), an impact of each of the components in the water samples on water transparency based on data obtained from S1-S4, wherein
a percentage of water transparency degradation that the large suspended particles account for is given by:
$$\frac{lx 2 - lx 1}{lx 0 - lx 1} ;$$
a percentage of water transparency degradation that the small inorganic suspended particles account for is given by:
$$\frac{lx 0 - lx 4}{lx 0 - lx 1} ;$$
a percentage of water transparency degradation that the dissolved colored substances account for is given by:
$$\frac{lx 0 - lx 3}{lx 0 - lx 1} ;$$
and
a percentage of water transparency degradation that the algae account for is given by:
$$\frac{( lx 3 - lx 2 ) - ( lx 0 - lx 4 )}{lx 0 - lx 1} .$$
